(19) United States
(12) Patent Application Publication
(10) Pub. No.: US 2017/0079604 A1
(43) Pub. Date: Mar. 23, 2017

(54) SYSTEM AND METHOD FOR DIGITAL BREAST TOMOSYNTHESIS

(71) Applicant: Anbinh T. Ho, Lewisville, TX (US)

(72) Inventor: Anbinh T. Ho, Lewisville, TX (US)

(21) Appl. No.: 15/269,922

(22) Filed: Sep. 19, 2016

Related U.S. Application Data
(60) Provisional application No. 62/220,745, filed on Sep. 18, 2015.

(51) Int. Cl. A61B 6/00; A61B 10/00; G06K 9/46; A61B 6/02; G06T 7/00; G06K 9/62

(52) U.S. Cl. CPC A61B 6/502; G06T 7/0012; G06K 9/6201; G06K 9/4604; A61B 6/025; A61B 6/5217; A61B 6/5223; A61B 10/0041; G06T 2207/10112; G06T 2207/30068; G06T 2207/30096; G06T 2207/10116

(57) ABSTRACT

An embodiment method for identifying anomalies in an object includes detecting bulges in a 2D image of the object, detecting 2D convergences in the 2D image of the object, detecting 3D convergences in each of a plurality of tomography slices of the object, and removing convergence overlap from overlapping 2D convergences to generate non-overlapping 2D convergences. For each non-overlapping 2D convergence, the method includes determining whether there is a matching 3D convergence in each slice of the plurality of tomography slices. Then for each matching 3D convergence for each non-overlapping 2D convergence, the method includes determining feature values of features of the matching 3D convergence. The method includes generating mass composite signatures in accordance with the detected bulges, the detected 2D convergences, and the feature values of the matching 3D convergences, and generating marks indicating the anomalies on the 2D image.

### RELATED APPLICATION

This application clams the benefit of co-pending U.S. Provisional Application Ser. No. 62/226,745, entitled SYSTEM AND METHOD FOR DIGITAL BREAST TOMOSYNTHESIS (DBT), filed Sep. 18, 2015, the teachings of which are expressly incorporated herein by reference.

### FIELD OF INVENTION

The present invention relates generally to a system and method for computer aided detection (CAD), and, in particular embodiments, to a system and method for digital breast tomosynthesis (DBT) CAD.

### BACKGROUND INFORMATION

Radiologists use radiographic images such as mammograms to detect and pinpoint suspicious lesions in a patient as early as possible, e.g., before a disease is readily detectable by other, intrusive methods. As such, there is real benefit to the radiologist being able to locate, based on imagery, extremely faint lesions and precursors. Large masses of relatively dense tissue are one signature of concern. Although some masses can appear quite prominent in a radiographic image, various factors including occlusion/partial occlusion by other natural structure, appearance in a structurally “busy” portion of the image, sometimes coupled with radiologist fatigue, may make some masses hard to detect upon visual inspection. One thing that can help identify a suspicious mass, particularly when its central bulge is difficult to see, is a spiculation pattern surrounding the mass. The spiculation pattern can appear in a radiographic image as a pattern of tissue that appears “drawn in” toward a central point.

Computer-aided detection (CAD) algorithms have been developed to assist radiologists in locating potential lesions in a radiographic image. CAD algorithms operate within a computer on a digital representation of the mammogram set for a patient. The digital representation can be the original or processed sensor data, when the mammograms are captured by a digital sensor, or a scanned version of a traditional film-based mammogram set, An “mage,” as used herein, is assumed to be at least two-dimensional data in a suitable digital representation for presentation to CAD algorithms, without distinction to the capture mechanism originally used to capture patient information. The CAD algorithms search the image for objects matching a signature of interest, and alert the radiologist when a signature of interest is found.

### SUMMARY OF THE INVENTION

An embodiment method for identifying anomalies in an object includes detecting bulges in a two-dimensional (2D) image of the object, detecting 2D convergences in the 2D image of the object, detecting three-dimensional (3D) convergences in each of a plurality of tomography slices of the object, and removing convergence overlap from overlapping 2D convergences to generate non-overlapping 2D convergences. For each non-overlapping 2D convergence, the method includes determining, whether there is a matching 3D convergence in each slice of the plurality of tomography slices. Then for each matching 3D convergence for each non-overlapping 2D convergence, the method includes determining feature values of features of the matching 3D convergence. The method further includes generating mass composite signatures in accordance with the detected bulges, the detected 2D convergences, and the feature values of the matching 3D convergences, and generating marks indicating the anomalies on the 2D image in accordance with the mass composite signatures.

An embodiment system for computer aided detection of anomalies in an object includes a processor and a non-transitory computer readable storage medium storing programming for execution by the processor. The programming includes instructions for performing the steps of the above method.

An embodiment apparatus for identifying anomalies in an object includes a non-transitory computer-readable memory and a hardware processor operably coupled to the memory. The hardware processor in conjunction with the memory is configured for performing the steps of the above method.

### BRIEF DESCRIPTION OF THE DRAWINGS

For a more complete understanding of the present invention, and the advantages thereof, reference is now made to the following descriptions taken in conjunction with the accompanying drawings. In the drawings, like reference, numbers are used herein to designate like or similar elements throughout the various views of illustrative embodiments. The figures are not necessarily drawn to scale, and in some instances the drawings have been exaggerated and/or simplified in places for illustrative purposes only. One of ordinary skill in the art will appreciate the many possible applications and variations based on the illustrative embodiments. In the drawings:

FIG. 1 is a system-level diagram for an anomaly detection system in accordance with an embodiment;

FIG. 2 is a component diagram of a CAD unit in accordance with an embodiment;

FIG. 3 is a component diagram of a detection writ in accordance with an embodiment;

FIG. 4 is a system block diagram for an overall CAD process;

FIG. 5 is a system block diagram for an overall CAD process utilizing DBT;

FIG. 6 is a sample output of a pairing process for each 2D convergence detection;

FIG. 7 is a sample output containing the feature value of the matching detection of each slice;

FIG. 8 is a block diagram of the generate mass composites block of FIG. 5 and

FIG. 9 is a block diagram of a computing device in accordance with an embodiment.

### DETAILED DESCRIPTION

The making and using of embodiments are discussed in detail below. It should be appreciated, however, that the present invention provides many applicable inventive concepts that can be embodied in a wide variety of specific contexts. The specific embodiments discussed are merely illustrative of specific ways to make and use the invention, and do not limit the scope of the invention.

For example, embodiments discussed herein are generally described in terms of assisting medical personnel in the examination of breast x-ray images, such as those that may be obtained in the course of performing a mammogram. Other embodiments, however, may be used for other situations, including, for example, detecting anomalies in other tissues such as lung tissue, any type of image analysis for statistical anomalies, and the like.

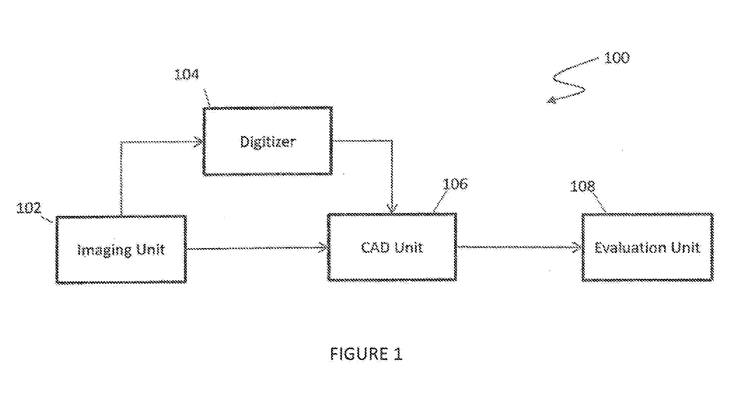

U.S. Pat. No. 8,923,594, issued on Dec. 30, 2014, which patent is hereby incorporated herein by reference, describes a system and method for malignant mass detection and classification in radiographic image. As described in this patent, FIG. 1 illustrates a system 100 for assisting in detecting anomalies during, for example, mammograms, is illustrated in accordance with an embodiment. The system 100 includes an imaging unit 102, a digitizer 104 and a computer-aided detection (CAD) unit 106. The imaging unit 102 captures one or more images, such as x-ray images, of the area of interest, such as the breast tissue. In the embodiment in which the system 100 is used to assist in analyzing a mammogram, a series of four x-ray images may be taken while the breast is compressed to spread the breast tissue, thereby aiding in the detection of anomalies. The series of four x-ray images include a top-down image, referred to as a cranio caudal (CC) image, for each of the right and left breasts, and an oblique angled image taken from the top of the sternum angled downwards toward the outside of the body, referred to as the medio lateral oblique (MLO) image, for each of the right and left breasts.

The one or more images may be embodied on film or digitized. Historically the one or more images are embodied as x-ray images on film, but current technology allows for x-ray images to be captured directly as digital images in much the same way as modem digital cameras. As illustrated in FIG. 1, a digitizer 104 allows for digitization of film images into a digital format. The digital images may be formatted in any suitable format, such as industry standard Digital imaging and Communications in Medicine (DICOM) format.

The digitized images, e.g., the digitized film images or images captured directly as digital images, are provided to a CAD unit 106. As discussed in greater detail below, the CAD unit 106 processes the one or more images to detect possible locations of various types of anomalies, such as calcifications, relatively dense regions, distortions, and/or the like. Once processed, locations of the possible anomalies, and optionally the digitized images, are provided to an evaluation unit 108 for viewing by a radiologist, the attending doctor, or other personnel with or without markings indicating positions of any detected possible anomalies. The evaluation unit 108 may comprise a display, a workstation, portable device, and/or the like.

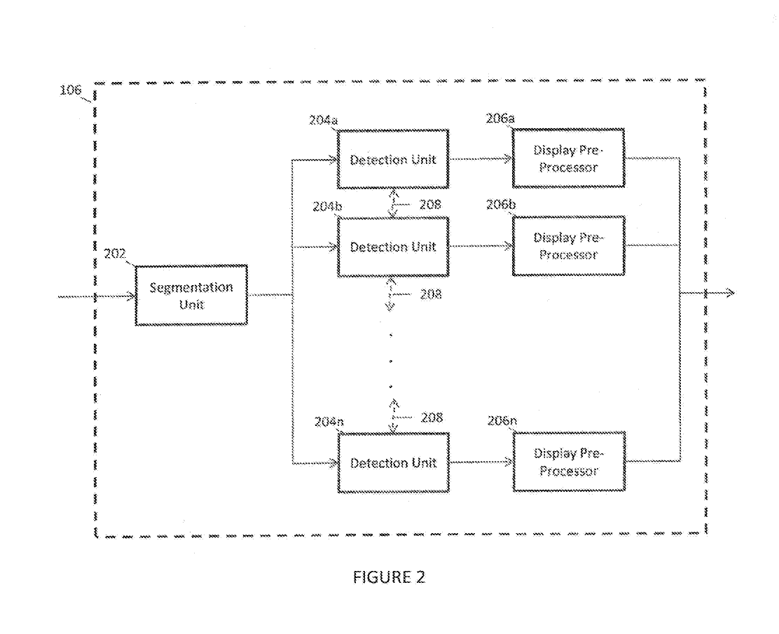

FIG. 2 illustrates components that may be utilized by the CAD unit 106 (see FIG. 1) in accordance with an embodiment. Generally, the CAD unit 106 includes a segmentation unit 202, one or more detection units 204a-204n, and one or more display pre-processors 206a-206n. As will be appreciated, an x-ray image, or other image, may include regions other than those regions of interest. For example, an x-ray image of a breast may include background regions as well as other structural regions such as the pectoral muscle. In these situations, it may be desirable to segment the x-ray image to define a search area, e.g., a bounded region defining the breast tissue, on which the one or more detection units 204a-204n is to analyze for anomalies.

The one or more detection units 204a-204n analyze the one or more images, or specific regions as defined by the segmentation unit 202, to detect specific types of features that may indicate one or more specific types of anomalies in the patient. For example, in an embodiment for use in examining human breast tissue, the detection units 204a-204n may comprise a calcification unit, a density (mass) unit, and a distortion unit. As is known in the medical field, the human body often reacts to cancerous cells by surrounding the cancerous cells with calcium, creating micro-calcifications. These micro-calcifications may appear as small, bright regions in the x-ray image. The calcification unit detects and identifies these regions of the breast as possible micro-calcifications.

It is further known that cancerous regions tend to be denser than surrounding tissue, so a region appearing as a generally brighter region indicating denser tissue than the surrounding tissue may indicate a cancerous region. Accordingly, the density unit analyzes the one or more breast x-ray images to detect relatively dense regions in the one or more images. Because the random overlap of normal breast tissue may sometimes appear suspicious, in some embodiments the density unit may correlate different views of an object, e.g., a breast, to determine if the dense region is present in other corresponding views. if the dense region appears in multiple views, then there is a higher likelihood that the region is truly malignant.

The distortion unit detects structural defects resulting from cancerous cells effect on the surrounding tissue. Cancerous cells frequently have the effect of “pulling in” surrounding tissue, resulting in spiculations that appear as a stretch mark, star pattern, or other linear line patterns.

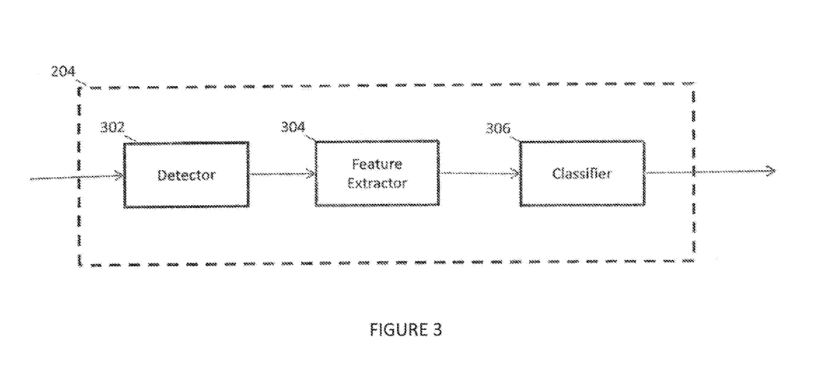

It should be noted that the above examples of the detection units 204a-204n, e.g., the calcification unit, the density unit, and the distortion unit, are provided for illustrative purposes only and that other embodiments may include more or fewer detection units, it should also be noted that some detection units may interact with other detection units, as indicated by the dotted line 208. The detection units 204a-204n are discussed in greater detail below with reference to FIG. 3.

The display pre-processors 206a-206n create image data to indicate the location and/or the type of anomaly. For example, micro-calcifications may be indicated by a line encircling the area of concern by one type of line (e.g., solid lines), while spiculations (or other type of anomaly) may be indicated by to line encircling the area of concern by another type of line (e.g., dashed lines).

FIG. 3 illustrates components of that may be utilized for each of the detection units 204a-204n in accordance with an embodiment. Generally, each of the detection units 204a-204n may include a detector 302, a feature extractor 304, and a classifier 306. The detector 302 analyzes the image to identify attributes indicative of the type of anomaly that the detection unit is designed to detect, such as calcifications, and the feature extractor 304 extracts predetermined features of each detected region. For example, the predetermined features may include the size, the signal-to-noise ratio, location, and the like.

The classifier 306 examines each extracted feature from the feature extractor 304 and determines a probability that the extracted feature is an abnormality. Once the probability is determined, the probability is compared to a threshold to determine whether or not a detected region is to be reported as a possible area of concern.

With respect to the system as described thus far, a suitable segmentation unit 202 is described in U.S. Pat. No. 8,675,934, entitled “Breast Skirt Line Detection in Radiographic Images,” issued, on Mar. 18, 2014, and U.S. Pat. No. 8,675,933, entitled “Breast Segmentation in Radiographic Images,” issued on Mar. 18, 2014; a suitable detection unit 204 for use in detecting and classifying micro-calcifications is described in U.S. Pat. No. 8,855,388, entitled “Micro-calcification Detection Classification In Radiographic Images,’ issued on Oct. 7, 2014; a suitable detection unit 204 for detecting and classifying malignant masses is described in U.S. Patent Application Publication No. 2013/0202165, entitled “Malignant Mass Detection and Classification in Radiographic Images,” published on Aug. 8, 2013; a suitable probability density function estimator for use in classifier 306 is described in U.S. Pat. No. 9,076,197, entitled “Probability Density Function Estimation,” issued on Jul. 7, 2015; and a suitable display pre-processor is described in U.S. Patent Application Publication No. 2013/0109953, entitled “Marking System for Computer-Aided Detection of Breast Abnormalities,” published on May 2, 2013, all of which patents and publications are hereby incorporated herein by reference, and all of which are usable in conjunction with various embodiments described below.

The following discussion provides greater details regarding a malignant mass detection unit, such as may be utilized as one or more of the detection units 204a-204n (see FIG. 2) in accordance with an embodiment. In particular, the embodiments described below seek to detect and classify potentially malignant masses in a radiographic image.

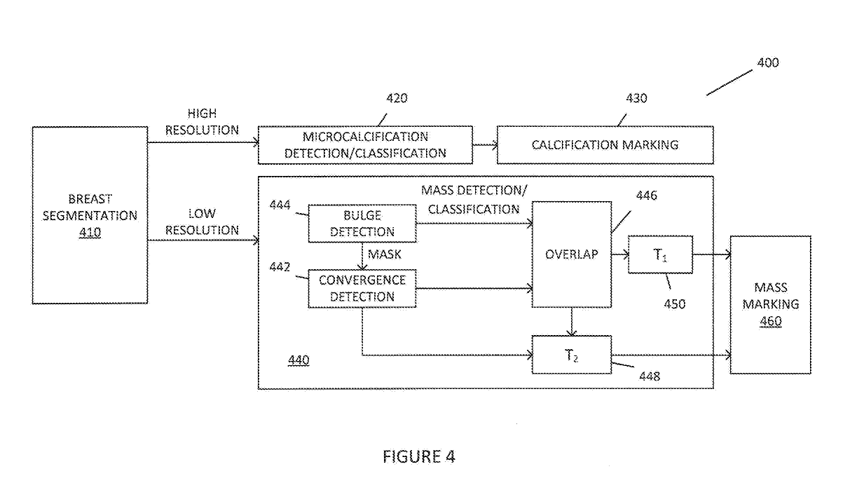

As a system overview, FIG. 4 illustrates system block diagram 400 for a detection/classification process useful with mammography imaging systems as described in U.S. Pat. No. 8,923,594. In FIG. 4, breast segmentation process 410 attempts to distinguish breast tissue from non-breast tissue areas of a mammogram. Breast segmentation 410 passes a breast mask image and a high-resolution version of the radiographic image to a micro-calcification detection/classification stage 420, which seeks to find clusters of micro-calcifications indicative of malignancy. When such clusters are found, descriptions for their location and extent are passed to calcification marking 430, which merges overlapping clusters as appropriate, and generates marks as needed for CAD result printouts, storage, display to a radiologist on an overlay of the displayed mammogram, etc.

Breast segmentation 410 also passes the breast mask image and a low-resolution version of the radiographic image to a mass detection/classification stage 440. A bulge detection unit 444 searches the lower resolution image for extended mass-like features at various scales.

The locations of bulges detected at any of the scales are passed as a mask to a convergence detection unit 442. Convergence detection unit 442 searches the lower resolution image for converging image features indicative of spiculated masses, at the locations of bulges indicated by the bulge detection unit.

Mass detection/classification stage 440 performs classification on the most significant bulges and convergences located in the image. An overlap mapper 446 determines whether each significant bulge and convergence pair overlap to an extent that the hub of the convergence appears to be co-located with the bulge, in which case an additional joint classification is made for the combined bulge/convergence. Overlap detector 446 passes each bulge or bulge/convergence pair, along with its probability of malignancy, to a probability threshold detector 450, and passes each convergence that does not overlap one of the bulges to a probability threshold detector 448. The different threshold detectors allow, e.g., a convergence that is not confirmed by a significant co-located bulge to be deemed suspicious at a higher threshold than other mass detections. Threshold detectors 448 and 450 pass the locations and extent of suspicious masses to a mass marking stage 460. Mass marking stage 460 merges overlapping clusters as appropriate, and generates marks as needed for CAD result printouts, storage, display to a radiologist on an overlay of the displayed mammogram, etc. Further details of the convergence detection process are provided in U.S. Pat. No. 8,923,594.

Embodiments described herein provide tomosynthesis CAD, which may be utilized in conjunction with the two-dimensional (2D) convergence detection of system 400 shown in FIG. 4, or in conjunction with other systems that would benefit from combined 2D and three-dimensional (3D) image processing. Digital breast tomosynthesis (DBT) provides high-resolution limited-angle tomography at mammographic dose levels. As disclosed herein, DBT combined with 2D convergence detection can provide a higher diagnostic accuracy compared to conventional mammography and other approaches.

As used herein, and unless otherwise obvious from a given context, 2D generally refers to for-processing, image(s), and 3D or tomo refer to tomosynthesis reconstructed image(s).

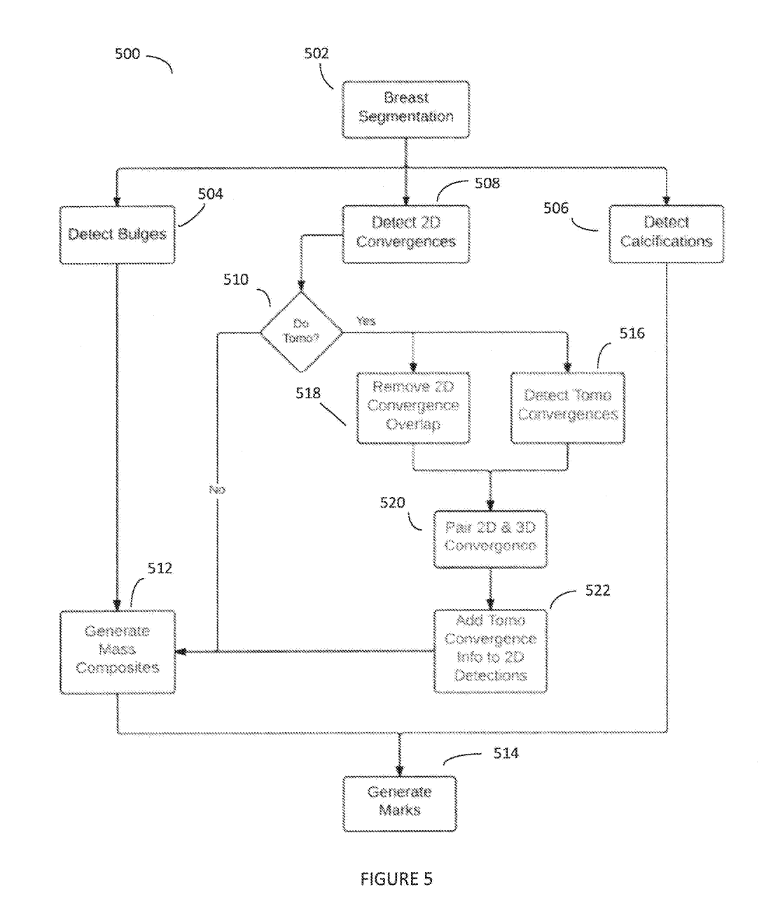

FIG. 5 illustrates an overview of an embodiment CAD method 500, which in one embodiment incorporates DBT into the system of FIG. 4. Breast segmentation 502, bulge detection 504 and calcification detection 506 are performed as described with respect to FIG. 4. After 2D convergences are detected 508, a decision 510 is made as to whether to proceed only with the detected 2D convergences, or to generate tomo convergences as well. If only 2D convergences are to be used, then generation of mass composites 512 is performed on the detected bulges and 2D convergences, and generation of marks 514 is performed using the mass composites and detected calcifications, as described above with respect to FIG. 4.

If tomo is selected, detect tomo convergences 516 finds convergence detections on each tomo slice using breast segmentation and convergence detection methods described and referenced above. A log likelihood ratio (LLR) is determined for each detection using probability distribution functions (PDFs) methods described and referenced above. The mass map generated from the 2D bulge detection described/referenced above is reused. The coordinate system generated from the 2D images described/referenced above is reused. The map of large bright objects generated from the 2D images described/referenced above is reused.

Remove 213 convergence overlap 518 selects one convergence detection among overlapping 2D convergences. The detection with the highest malignant LLR is selected. Starting with the most malignant detection, the method defines a square of a certain size centered around the detection's centroid, and invalidates all detections with centroids in the square. This is repeated for the highest malignant LLR of the remaining detections.

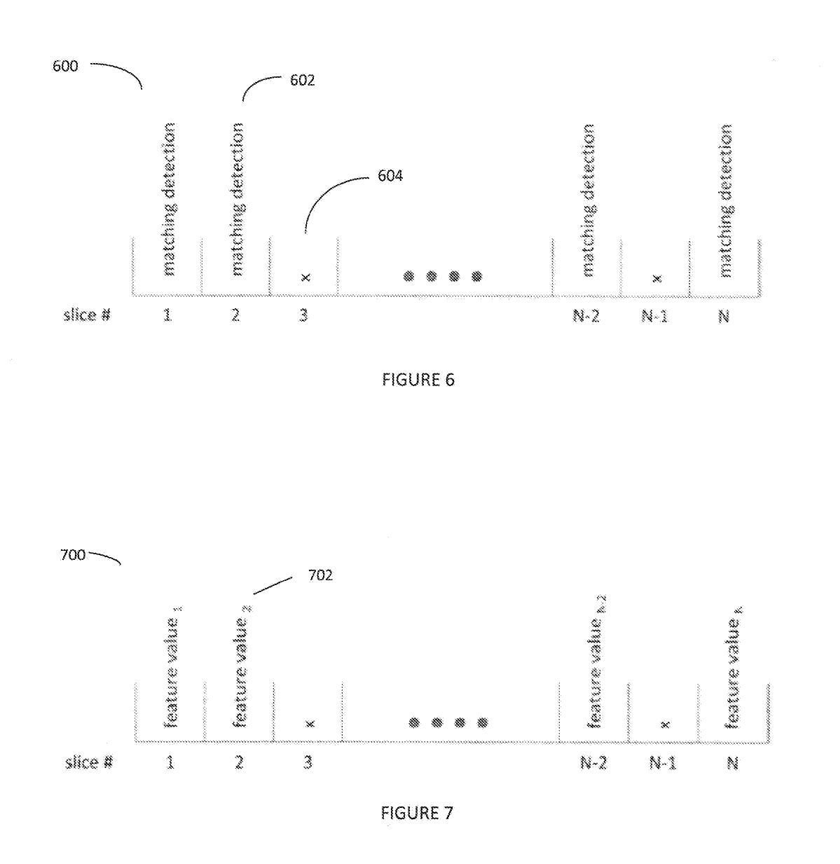

For each non-overlapping 2D convergence detection, pair 2D & 3D convergence 520 defines an appropriate search area as well as a set of detection-matching criteria. For each tomosynthesis slice, one convergence detection is found that meets all the specified matching criteria defined by the 2D convergence under consideration, as well as any tie-breaker criteria. It is possible for the search to produce an empty result. FIG. 6 illustrates an example sample output 600 generated by the pairing process for each 2D convergence detection, indicating matching detections 602 and non-matching detections 604.

With reference back to FIG. 5, add tomo convergence information to 2D detection 522 extracts various features and properties of the matching 3D detections, for each non-overlapping 2D convergence detection. For each feature/property, the output is an array with a length equal to the number of slices. Each array element contains the feature value of the matching detection for that slice. If the slice contains no matching detections, the feature value is set to zero. Different matching criteria can be applied to the pairing process for different features/properties. FIG. 7 illustrates an example sample output 700 containing the feature value 702 of the matching detection of each slice. The results are then utilized, along with results of bulge detection 504, to generate mass composites 512. Generate marks 514 is then performed using the mass composites and detected calcifications.

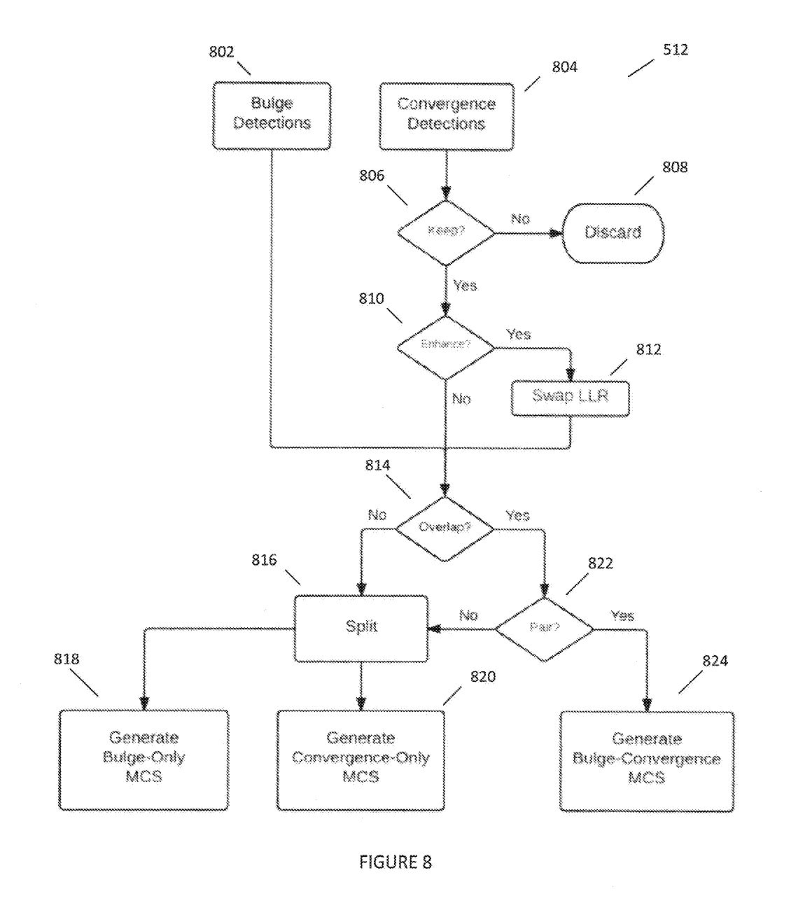

An overview of the generate mass composites process 512 is shown in FIG. 8. Bulge detections 802 are from detect bulges 504, and convergence detections 804 are from detect 2D convergences 508 or add tomo convergence information to 2D detections 522.

The following two concepts apply to each 2D convergence detection. First, a slice window attempts to identify a slice range where the object of interest is visible. This is done by identifying consecutive slices that contain matching 3D convergence detections. Each 2D convergence detection may have zero to multiple slice window(s). Second, signal significance determines if the feature value(s) in the current slice window are significant when compared to the feature values outside the window.

Keep 806 determines whether a 2D convergence detection should be kept or discarded 808. Generally, if the 2D detection came from a real signal (object), as opposed to overlapping noise, then there should exist some 3D slices where the signal from the object (e.g., lesion) is very strong. The signal also should be stronger than slices that do not contain the object.

In one embodiment, a 2D convergence detection is kept if the following criteria are met:

(1) a valid slice window is found,

(2) the maximum signal strength in the window is higher than the signal strength in the 2D image, and

(3) the maximum signal strength in the window is significant.

Alternatively, a 2D convergence detection is kept if the following criteria are met:

(1) a valid slice window is found, and

(2) the maximum signal strength in the window is higher than a defined threshold.

If a 2D convergence detection is kept, enhance 810 determines if the 2D convergence detection should have its LLR replaced by a higher LLR found in the 3D slices. Generally, the presence of a strong and unusual signal suggests a real cancer-like object. When forming a mass composite detection with this 2D convergence detection, the strongest malignant LLR detected by the system is used.

Index alignment is checked between the slice with the highest signal strength and the slice with the highest malignant LLR. If the indices align, then the enhancement criteria are checked using the maximum signal strength in the window and the maximum malignant LLR in the window. If the indices do not align, the enhancement criteria are checked using the signal strength and the malignant LLR value at the index where the maximum signal strength occurs. Then, the enhancement criteria is checked using the values at the index where the maximum malignant LLR occurs. The criteria only need to be net at one location for enhancement to be triggered.

In one embodiment, a 2D convergence detection is enhanced if the following criteria are met:

(1) a valid slice window is found,

(2) the signal strength is higher than the signal strength in the 2D image,

(3) the signal strength is significant relative to the signal in the other slices, and

(4) the malignant LLR in the window is significant.

Alternatively, a 2D convergence detection is enhanced if the following criteria are met:

(1) a valid slice window is found,

(2) the signal strength in the window is higher than a defined threshold, and

(3) the malignant LLR in the window is significant.

If the 2D convergence detection is determined to be enhanced, then swap LLR 812 replaces the 2D convergence with the maximum between the 2D and all eligible 3D convergence LLRs.

Overlap 814 determines whether there is overlap between a bulge detection and a convergence (enhanced or non-enhanced) detection. If no overlap is determined, then the bulge detection and the convergence detection are split 816. Generate bulge-only mass composite signature (MCS) 818 generates a bulge-only MCS for a detected bulge, and generate convergence-only MCS 820 generates a convergence-only mass composite signature for the (enhanced or non-enhanced) convergence detection.

If overlap 814 determines there is an overlap between a bulge detection and a (enhanced or non-enhanced) convergence detection, then the two are further analyzed to determine whether pairing takes place. That is, pair 822 determines if a 2D convergence detection should be paired with any overlapping 2D bulge detection(s), Generally, convergence and bulge detections are paired if a significant convergence signal is found in a tomo slice range that also contains a significant indication of tissue density.

In an embodiment, convergence and bulge detections are paired if the following criteria are met:

(1) a valid slice window is found,

(2) the maximum malignant LLR in the window is significant, and

(3) the maximum contrast minimum in the window is significant.

Alternatively, convergence and bulge detections are paired if the following criteria are met:

(1) a valid slice window is found,

(2) the maximum malignant LLR in the window is significant, and

(3) the maximum contrast minimum in the window is higher than a define threshold.

Generate marks 514 (FIG. 5) takes the output of the generate mass composites 512 and the output of detect calcifications 506, and generates marks as desired for CAD result printouts, storage, display to a radiologist on an overlay of the displayed mammogram, etc., as discussed previously herein.

Unless indicated otherwise, all functions described herein may be performed in either hardware or software, or some combination thereof. In a preferred embodiment, however, the functions are performed by a processor such as a computer or an electronic data processor in accordance with code such as computer program code, software, and/or integrated circuits that are coded to perform such functions, unless otherwise indicated.

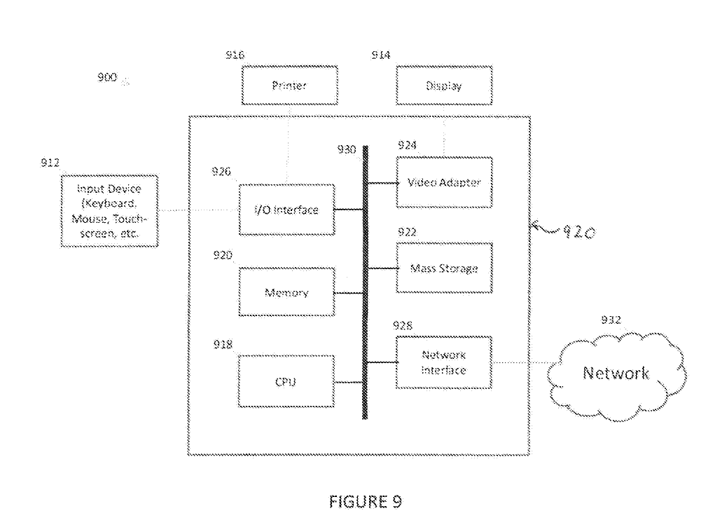

For example, FIG. 9 is a block diagram of a computing system 900 that may also be used in accordance with an embodiment. It should be noted, however, that the computing system 900 discussed herein is provided for illustrative purposes only and that other devices may be used. The computing system 900 may comprise, for example, a desktop computer, a workstation, a laptop computer, a personal digital assistant, a dedicated unit customized for a particular application, or the like. Accordingly, the components of the computing system 900 disclosed herein are for illustrative purposes only and other embodiments ma include additional or fewer components.

In an embodiment, the computing system 900 comprises a processing unit 910 equipped with one or more input devices 912 (e.g., a mouse, a keyboard, or the like), and one or more output devices, such as a display 914, a printer 916, or the like. Preferably, the processing unit 910 includes a central processing unit (CPU) 918, memory 920, a mass storage device 922, a video adapter 924, an I/O interface 926, and a network interface 928 connected to a bus 930. The bus 930 may be one or more of any type of several bus architectures including a memory bus or memory controller, a peripheral bus, video bus, or the like. The CPU 918 may comprise any type of electronic data processor. For example, the CPU 918 may comprise a processor (e.g., single core or multi-core) from Intel Corp. or Advanced Micro Devices, Inc., a Reduced Instruction Set Computer (RISC), an Application-Specific Integrated Circuit (ASIC), or the like. The memory 920 may comprise any type of system memory such as static random access memory (SRAM), dynamic random access memory (DRAM), synchronous DRAM (SDRAM), read-only memory (ROM), a combination thereof, or the like. In an embodiment shown in FIG. 9, the memory 920 may include ROM for use at boot-up, and DRAM for data storage for use while executing programs. The memory 920 may include one of more non-transitory memories.

The mass storage device 922 may comprise any type of storage device configured to store data, programs, and other information and to make the data, programs, and other information accessible via the bus 928. In an embodiment, the mass storage device 922 is configured to store the program to be executed by the CPU 918. The mass storage device 922 may comprise, for example, one or more of a hard disk drive, a magnetic disk drive, an optical disk drive, or the like. The mass storage device 922 may include one or more non-transitory memories.

The video adapter 924 and the I/O interface 926 provide interfaces to couple external input and output devices to the processing unit 910. As illustrated in FIG. 9, examples of input and output devices include the display 614 coupled to the video adapter 924 and the mouse/keyboard 912 and the printer 916 coupled to the interface 926. Other devices may be coupled to the processing unit 910.

The network interface 928, which may be to wired link and/or a wireless link, allows the processing unit 910 to communicate with remote units via the network 932. In an embodiment, the processing unit 910 is coupled to a local-area network or a wide-area network to provide communications to remote devices, such as other processing units, the Internet, remote storage facilities, or the like

It should be noted that the computing system 900 may include other components. For example, the computing system 900 may include power supplies, cables, a motherboard, removable storage media, cases, a network interface, and the like. These other components, although not shown, are considered part of the computing system 900. Furthermore, it should be noted that any one of the components of the computing system 900 may include multiple components. For example, the CPU 918 may comprise multiple processors, the display 914 may comprise multiple displays, and/or the like. As another example, the computing system 900 may include multiple computing systems directly coupled and/or networked.

Additionally, one or more of the components may be remotely located. For example, the display may be remotely located from the processing unit, In this embodiment, display information, e.g., locations and/or types of abnormalities, may be transmitted via the network interface to a display unit or a remote processing unit having a display coupled thereto.

Although several embodiments and alternative implementations have been described, many other modifications and implementation techniques will be apparent to those skilled in the art upon reading this disclosure. Various parameters and thresholds exist and can be varied for a given implementation with given data characteristics, with experimentation and ultimate performance versus computation time tradeoffs necessary to arrive at a desired operating point. Although at least one specific method has been described for calculation of each feature type, many alternate methods and feature definitions exist for calculating similar features with similar or acceptable performance. For example, various embodiments use a PDF-classification implementation with the feature set. It is believed that the disclosed feature set, convergence and DBT techniques can also be advantageous in CAD systems not using a PDF-classification approach.

Although the specification may refer to “an”, “one”, “another”, or “some” embodiment(s) in several locations, this does not necessarily mean that each such reference is to the same embodiment(s), or that the feature only applies to a single embodiment.

What is claimed is:

1. A method for identifying anomalies in an object, the method comprising:
detecting bulges in a two-dimensional (2D) image of the object;
detecting 2D convergences in the 2D image of the object;
detecting three-dimensional (3D) convergences in each of a plurality of tomography slices of the object;
removing convergence overlap from overlapping 2D convergences to generate non-overlapping 2D convergences;
for each non-overlapping 2D convergence, determining whether there is a matching 3D convergence in each slice of the plurality of tomography slices;
for each matching 3D convergence for each non-overlapping 2D convergence, determining feature values of features of the matching 3D convergence;
generating mass composite signatures in accordance with the detected bulges, the detected 2D convergences, and the feature values of the matching 3D convergences; and
generating marks indicating, the anomalies on the 2D image in accordance with the mass composite signatures.

2. The method of claim 1, wherein generating the mass composite signatures comprises, for each non-overlapping 2D convergence:
determining a slice window of consecutive tomography slices each having a matching 3D convergence for the non-overlapping 2D convergence; and
determining whether to keep or discard the non-overlapping 2D convergence in accordance with a maximum signal strength within the slice window.

3. The method of claim 2, wherein generating the mass composite signatures further comprises, for each kept non-overlapping 2D convergence:
determining whether to enhance a malignant log likelihood ratio (LLR) of the kept non-overlapping 2D convergence in accordance with a signal strength within the slice window and a malignant LLR within the slice window; and
using, as an updated malignant LLR of the kept non-overlapping 2D convergence. a maximum malignant LLR selected from the kept non-overlapping 2D convergence and the matching 3D convergences in the slice window.

4. The method of claim 3, further comprising:
determining whether there is overlap between the updated malignant LLR of the kept non-overlapping 2D convergence and any of the detected bulges.

5. The method of claim 4, wherein generating the mass composite signatures comprises, in response to determining there is no overlap between the updated malignant LLR of the kept non-overlapping 2D convergence and any of the detected bulges:
generating a convergence mass composite signature for the kept non-overlapping 2D convergence separately from generating mass composite signatures for the detected bulges.

6. The method of claim 4, further comprising, in response to determining there is overlap between the updated malignant LLR of the kept non-overlapping 2D convergence and one of the detected bulges:
determining whether to pair the kept non-overlapping 2D convergence and the one of the detected bulges in accordance with a maximum malignant LLR in the slice window and a maximum contrast minimum in the slice window.

7. The method of claim 6, wherein generating the mass composite signatures comprises, in response to determining to pair the kept non-overlapping 2D convergence and the one of the detected bulges:
generating a combined bulge and convergence mass composite signature for the kept non-overlapping 2D convergence and the one of the detected bulges.

8. A system for computer aided detection of anomalies in an object, the system comprising:
a processor; and
a non-transitory computer readable storage medium storing programming for execution by the processor, the programming including instructions for:
detecting bulges in a two-dimensional (2D) image of the object;
detecting 2D convergences in the 2D image of the object;
detecting three-dimensional (3D) convergences in each of a plurality of tomography slices of the object;
to removing convergence overlap from overlapping 2D convergences to generate non-overlapping 2D convergences;
for each non-overlapping 2D convergence, determining whether there is a matching 3D convergence in each slice of the plurality of tomography slices;
for each matching 3D convergence for each non-overlapping 2D convergence, determining feature values of features of the matching 3D convergence;
generating mass composite signatures in accordance with the detected bulges, the detected 2D convergences, and the feature values of the matching 3D convergences; and
generating marks indicating the anomalies on the 2D image in accordance with the mass composite signatures.

9. The system of claim 8, wherein the instructions for generating the mass composite signatures comprise instructions for, for each non-overlapping 2D convergence:
determining a slice window of consecutive tomography slices each having a matching 3D convergence for the non-overlapping 2D convergence; and
determining whether to keep or discard the non-overlapping 2D convergence in accordance with a maximum signal strength within the slice window.

10. The system of claim 9, wherein the instructions for generating the mass composite signatures further comprise instructions for, for each kept non-overlapping 2D convergence:
determining whether to enhance as malignant log likelihood ratio (LLR) of the kept non-overlapping 2D convergence in accordance with a signal strength within the slice window and a malignant within the slice window; and
using, as an updated malignant LLR of the kept non-overlapping 2D convergence, a maximum malignant LLR selected from the kept non-overlapping 2D convergence and the matching 3D convergences in the slice window.

11. The system of claim 10, wherein the programming further comprises instructions for:
determining whether there is overlap between the updated malignant LLR of the kept non-overlapping 2D convergence and any of the detected bulges.

12. The system of claim 11, wherein the instructions for generating the mass composite signatures comprise instructions for, in response to determining there is no overlap between the updated malignant LLR of the kept non-overlapping 2D convergence and any of the detected bulges:
generating a convergence mass composite signature for the kept non-overlapping 2D convergence separately from generating mass composite signatures for the detected bulges.

13. The system of claim 11, wherein the programming further comprises instructions for, in response to determining there is overlap between the updated malignant LLR of the kept non-overlapping 2D convergence and one of the detected bulges:
determining whether to pair the kept non-overlapping 2D convergence and the one of the detected bulges in accordance with a maximum malignant LLR in the slice window and a maximum contrast minimum in the slice window.

14. The system of claim 13, wherein the instructions for generating the mass composite signatures comprise instructions for, in response to determining to pair the kept non-overlapping 2D convergence and the one of the detected bulges:
generating a combined bulge and convergence mass composite signature for the kept non-overlapping 2D convergence and the one of the detected bulges.

15. An apparatus for identifying anomalies in an object, the apparatus comprising:
a non-transitory computer-readable memory; and
a hardware processor operably coupled to the memory, the hardware processor in conjunction with the memory configured for:
detecting bulges in a two-dimensional (2D) image of the object;
detecting 2D convergences—in the 2D image of the object;
detecting three-dimensional (3D) convergences in each of a plurality of tomography slices of the object;
removing convergence overlap from overlapping 2D convergences to to generate non-overlapping 2D convergences;
for each non-overlapping 2D convergence, determining whether there is a matching 3D convergence in each slice of the plurality of tomography slices;
for each matching 3D convergence for each non-overlapping 2D convergence, determining feature values of features of the matching 3D convergence;
generating mass composite signatures in accordance with the detected bulges, the detected 2D convergences, and the feature values of the matching 3D convergences; and
generating marks indicating the anomalies on the 2D image in accordance with the mass composite signatures.

16. The apparatus of claim 15, wherein generating the mass composite signatures comprises, for each non-overlapping 2D convergence:
determining a slice window of consecutive tomography slices each having a matching 3D convergence for the non-overlapping 2D convergence; and
determining whether to keep or discard the non-overlapping 2D convergence in accordance with a maximum signal strength within the slice window.

17. The apparatus of claim 16, wherein generating the mass composite signatures further comprises, for each kept non-overlapping 2D convergence:
determining whether to enhance a malignant log likelihood ratio (LLR) of the kept non-overlapping 2D convergence in accordance with a signal strength within the slice window and a malignant LLR within the slice window; and
using, as an updated malignant LLR of the kept non-overlapping 2D convergence, a maximum malignant LLR selected from the kept non-overlapping 2D convergence and the matching 3D convergences in the slice window.

18. The apparatus of claim 17, the hardware processor in conjunction with the memory further configured for:
determining whether there is overlap between the updated malignant LLR of the kept non-overlapping 2D convergence and any of the detected bulges.

19. The apparatus of claim 18, wherein generating the mass composite signatures comprises, in response to determining there is no overlap between the updated malignant LLR of the kept non-overlapping 2D convergence and any of the detected bulges:
generating a convergence mass composite signature for the kept non-overlapping 2D convergence separately from generating mass composite signatures for the detected bulges.

20. The apparatus of claim 18, the hardware processor in conjunction with the memory further configured for, in response to determining there is overlap between the updated malignant LLR of the kept non-overlapping 2D convergence and one of the detected bulges:
determining whether to pair the kept non-overlapping 2D convergence and the one of the detected bulges in accordance with a maximum malignant LLR in the slice window and a maximum contrast minimum in the slice window.

21. The apparatus of claim 20, wherein generating the mass composite signatures comprises, in response to determining to pair the kept non-overlapping 2D convergence and the one of the detected bulges:
generating a combined bulge and convergence mass composite signature for the kept non-overlapping 2D convergence and the one of the detected bulges.
